(19) United States
(12) Patent Application Publication
(10) Pub. No.: US 2003/0138823 A1
(43) Pub. Date: Jul. 24, 2003

(54) Sample preparation methods for maldi mass spectrometry

(75) Inventors: Ansgar Brock, San Diego, CA (US); Qui Phung, San Diego, CA (US); Eric C. Peters, Carlsbad, CA (US)

Correspondence Address: QUINE INTELLECTUAL PROPERTY LAW GROUP, P.C., P O BOX 458, ALAMEDA, CA, 94501, US

(73) Assignee: IRM, LLC, Hamilton (BM)

(21) Appl. No.: 10/289,160

(22) Filed: Nov. 5, 2002

Related U.S. Application Data
(60) Provisional application No. 60/332,988, filed on Nov. 5, 2001.
(60) Provisional application No. 60/368,342, filed on Mar. 27, 2002.
(60) Provisional application No. 60/385,835, filed on Jun. 3, 2002.
(60) Provisional application No. 60/385,364, filed on Jun. 3, 2002.
(60) Provisional application No. 60/385,769, filed on Jun. 3, 2002.
(60) Provisional application No. 60/386,915, filed on Jun. 5, 2002.
(60) Provisional application No. 60/410,382, filed on Sep. 12, 2002.

(51) Int. Cl. C12Q 1/68

(52) U.S. Cl. 435/6

(57) ABSTRACT

This invention provides methods for preparing samples for MALDI mass spectrometry. The methods make possible the use of reagents that are normally considered unsuitable for MALDI in the preparation of a sample. Also provided are methods for internal calibration of a mass spectrometer, and methods for preparing sample supports.

### CROSS-REFERENCE TO RELATED APPLICATIONS

This application is related to U.S. provisional patent application U.S. S No. 60/386,915 filed Jun. 5, 2002. This application is also related to U.S. provisional patent applications U.S. S No. 60/332,988 filed Nov. 5, 2001; U.S. S No. 60/385,835 filed Jun. 3, 2002; and U.S. S No. 60/410,382 filed Sep. 12, 2002, titled “Labeling Reagent and Methods of Use”; as well as U.S. provisional patent applications U.S. S No. 60/368,342 filed Mar. 27, 2002; U.S. S No. 60/385,769 filed Jun. 3, 2002; and U.S. S No. 60/385,364 filed Jun. 3, 2002 and titled “Methods and Devices for Proteomics Data Complexity Reduction.” The present application claims priority to, and benefit of, these applications, pursuant to 35 U.S.C. §119(e) and any other applicable statute or rule.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to mass analysis. More particularly, the present invention relates to sample preparation, handling, and operating methods for mass spectrometry, and specifically to the automated sample processing, handling and operations as they relate to the technique of liquid chromatography matrix-assisted laser desorption mass spectrometry (LC-MALDI MS).

### BACKGROUND OF THE INVENTION

A number of sophisticated approaches have been developed to study the structure and function of genes, including the whole-scale sequencing of entire organisms, global transcriptional profiling, and forward genetic studies. However, these techniques are ultimately limited by the fact that they only assess intermediates on the way to the protein products of genes that ultimately regulate biological processes. Processes such as RNA processing, proteolytic activation, and hundreds of possible post-translational modifications (PTMs) can result in the production of numerous proteins of unique structure and function from a limited number of genes. Additionally, biological activity often results from the assembly of numerous proteins into an active complex, the nature and composition of which can only be explored at the protein level.

Proteomics is the study of the “proteome,” the protein complement expressed by a genome at a given point in time. Proteomic studies should be able to answer many questions about cellular processes and diseases that can't be answered by genomic methods alone. However, such studies are more difficult to perform than their genomic counterparts, and any general analysis platform must possess high sensitivity, be tolerant of a wide range of experimental and analytical conditions, and be able to process and display massive amounts of information. In addition, these analysis systems must also be able to perform extremely high-throughput measurements, since, unlike the relatively fixed nature of the genome, the expression and interactions of proteins are in a constant state of flux, varying over time, tissue type, and in response to environmental changes.

Historically, two-dimensional gel electrophoresis (2DE) has been the dominant technique for assessing large-scale changes in protein expression patterns. Although powerful, the 2DE technique as practiced remains laborious, and possesses several widely recognized limitations, including the difficulty of comparing results between laboratories, operational difficulty in handling certain classes of proteins, and potential unwanted chemical modifications. Another shortcoming of the classic 2DE technique is its inability to accommodate the extreme range of protein expression levels inherent in complex living organisms due to sample loading restrictions imposed by the gel-based separation technology employed.

The development and emergence of biological mass spectrometry (MS) in the early 1990's addressed some of these issues and greatly increased the amount of information obtained using two-dimensional gel electrophoresis, enabling the identification of thousands of encoded proteins by peptide mapping and/ or tandem MS experiments (for a general review see, for example, Karas and Hillenkamp (1988) “Laser desorption ionization of proteins with molecular masses exceeding 10,000 daltons” Anal. Chem. 60:2299-2301; Fenn et al. (1989) “Electrospray Ionization for Mass Spectrometry of Large Biomolecules” Science 246:64-71; and Patterson and Aebersold (1995) “Mass spectrometric approaches for the identification of gel-separated protein” Electrophoresis 16:1791-1814.

Multi-dimensional chromatography combined with MS and/or tandem MS methods has been explored as an alternative method to explore the proteome (see, for example, Yates (2000) “Mass spectrometry: from genomics to proteomics” Trends. Genet. 16:5-8; Aebersold and Goodlett (2001) “Mass spectrometry in proteomics” Chem. Rev. 101:269-95). Samples are partially purified and separated by one or more liquid chromatographic techniques, the fractions from which are then analyzed and identified by separating gaseous ions of the substances according to their mass-to-charge ratio. The chromatographic separations serve to disperse the complexity of the initial sample, and can be performed at both the peptide as well as at the protein level (although protein identification is typically performed using peptides). Electrospray ionization (ESI) methods are most commonly employed, due in part to the simplicity of their implementation. However, parameters for coupling LC and ESI mass spectrometry impose several undesirable limitations, making this technique less suitable for proteomics experiments. Another type of ionization used in mass spectrometry, matrix-assisted laser desorption/ionization (MALDI), has also been considered for proteomics studies; however, several shortcomings exist that also limit the use of MALDI/MS in proteomics. For example, the complex ionization processes resulting in the production of sample ions are not well understood. The process for preparing the matrix/analyte co-crystallizate has a profound impact on the signals that are detected from the sample; as such, a standard (and hopefully optimized) procedure is needed for preparing for MS analysis the plurality of fractionated sample components eluted under varying solvent conditions. In addition, a need exists for an efficient means to generate standardized sample substrates suitable for MALDI, optionally such that the plates can be used once and then regenerated. Furthermore, internal calibration is required to achieve and maintain highest performance under varying acquisition conditions.

The present invention addresses these shortcomings and other concerns in the art by providing novel methods for preparing samples and sample substrates for MALDI mass spectrometry while employing solvents generally considered to be MALDI-incompatible and/or internal calibrants.

### SUMMARY OF THE INVENTION

The present invention provides methods for preparing samples and sample substrates for mass spectrometry. In addition, the present invention provides sample substrates prepared by the methods herein, including solid supports having calibrant preloaded in specified positions. The samples and sample substrates prepared by the methods herein are particularly useful, for example, in automated sample processing for direct deposition liquid chromatography MALDI mass spectrometry and high mass measurement accuracy data collection.

Accordingly, the present invention provides novel methods for preparing a sample for MALDI spectrometry. The methods include the steps of a) spotting an aliquot of a matrix onto a MALDI support, wherein the matrix is optionally dissolved in a solvent generally considered to be MALDI-incompatible (i.e., a “MALDI-incompatible solvent”); b) depositing an analyte onto the MALDI support at a same position as the aliquot of the matrix; c) allowing the matrix and analyte to dry, thereby forming a co-crystallizate; d) depositing a recrystallization solution onto the co-crystallizate and redissolving the cocrystallizate; and e) allowing the redissolved co-crystallizate to dry, thereby forming a sample suitable for MALDI.

In the methods of the present invention, the steps of placing the matrix and the analyte onto the solid support can be performed in either order. Optionally, the support comprises hydrophilic target regions upon which the aliquots of the matrix and analyte are spotted. Exemplary MALDI-incompatible solvents for use in the methods of the present invention include, but are not limited to, chaotropic agents and/or low vapor pressure solvents. Either the matrix, the analyte, or both the matrix and the analyte can be prepared in the MALDI-incompatible solvent. Optionally, the MALDI-incompatible solvent also includes a sample solubilization agent. Exemplary sample solubilization agents include, but are not limited to, urea, various surfactants, and/or salts.

Typically, the recrystallization solution used in the second depositing step is a MALDI-compatible solvent, such as water, acetonitrile (ACN), acetone, ethanol, methanol, trifluoroacetic acid (TFA), and formic acid, or combinations thereof. Typically, the volume of recrystallization solution employed is less than the initial volume of matrix or analyte deposited onto the support. In some embodiments of the present invention, the depositing of recrystallization solution and subsequent drying steps are performed multiple times prior to submitting the sample for MALDI mass spectrometry. Optionally, the MALDI support is washed one or more times prior to depositing the recrystallization solution onto the co-crystallizate.

The present invention also provides novel methods for internal calibration of a mass spectrometer. The methods include the steps of a) providing a sample support that comprises an analyte at a first location on the support, and a calibrant at a second location on the support; b) ionizing the analyte and transiently storing analyte ions in an ion storage chamber; c) ionizing the calibrant and transiently storing calibrant ions in the ion storage chamber; and d) releasing a mixture of analyte ions and calibrant ions from the ion storage chamber into a mass analyzer. The ionization steps can be performed in either order (e.g. analyte first or calibrant first). Optionally, the ionization method is MALDI.

Transiently storing the analyte ions and the calibrant ions optionally involves trapping the ions with one or more multipole ion guides, trap electrodes, Penning traps, or a combination of ion trapping devices. In one embodiment of the methods, upon ionization, the analyte ions and the calibrant ions are guided using one or more ion optics elements to an entrance of a first mass analyzer, and passed through the first mass analyzer into the ion storage chamber. Optionally, mass selection is performed during this passage. In an another embodiment, only the analyte ions are guided to the entrance of the first mass analyzer and undergo mass selection, after which the selected analyte ions are combined with the calibrant ions in the ion storage chamber.

In the calibration methods of the present invention, the steps of ionizing the analyte and ionizing the calibrant can be performed in either order. In certain embodiments, the MALDI support is placed upon an adjustable stage movable along 2-dimensions (e.g., an x,y translational stage); the support is then moved such that the analyte and calibrant are sequentially positioned in line with the laser beam during the ionizing step. In an alternate embodiment, the laser beam is moveable to different positions on the MALDI sample support, in line with either the analyte or calibrant deposits.

Optionally, the ion storage chamber employed in the calibration methods includes an ion trap that provides for mass selection of the analyte and/or the calibrant. The ions can be transiently stored, e.g., by trapping the ions with one or more multipole ion guides, trap electrodes, Penning traps, or a combination of ion traps. Exemplary multipole ion guides for use in the ionizing and storing steps include, but are not limited to, a quadrupole ion guide, a hexapole ion guide, a octopole ion guide, a stacked ring ion guide, or a combination thereof. In some embodiments of the methods, transiently storing the analyte ions and/or the calibrant ions involves guiding the ions (using one or more ion optics elements) to an entrance of a first mass analyzer, and passing the ions through the first mass analyzer into the ion storage chamber. Optionally, the ions undergo mass selection during the passage.

In another embodiment, the calibration methods of the present invention include the steps of a) ionizing an analyte by a first method of ionization, transporting the analyte ions into an ion storage chamber through a first set of ion optical elements and transiently storing the analyte ions in the ion storage chamber; b) ionizing a calibrant by a second method of ionization, transporting the calibrant ions into an ion storage chamber through a second set of ion optical elements and transiently storing the calibrant ions in the ion storage chamber; and c) releasing the mixture of calibrant and analyte ions into a mass analyzer. Either the analyte ions or calibrant ions can be generated and stored first. The optical elements employed can be separate elements or the same set of elements (i.e., the first set of ion optical elements and second set of ion optical elements can be the same set).

In some embodiments, the ion source used for analyte ion generation is positioned first in front of ion optical elements transporting the analyte ions into the ion storage chamber, followed by positioning the ion source generating calibrant ions in front of the set of ion optical elements and transporting the calibrant ions second into the ion storage chamber. Alternatively, wherein an ion source used for calibrant ion generation is positioned first in front of the ion optical elements transporting the calibrant ions into the ion storage chamber first, followed by positioning an ion source generating analyte ions in front of the set of ion optical elements and transporting the analyte ions second into the ion storage chamber. Furthermore, the ionization steps can be performed using different ion sources, or even different ionization techniques.

Optionally, the ion storage chamber is part of the ionization region of the ion source generating analyte ions, or the ion source generating calibrant ions.

In an alternate embodiment of the calibration methods of the present invention, the sample and calibrant are provided on separate sample substrates. The methods of this embodiment include the steps of a) providing a first sample support that comprises an analyte and a second sample support that comprises a calibrant; b) ionizing the analyte using a laser beam and transiently storing analyte ions in an ion storage chamber; c) ionizing the calibrant using a laser beam and transiently storing calibrant ions in the ion storage chamber; and d) releasing a mixture of analyte ions and calibrant ions from the ion storage chamber into a mass analyzer.

The present invention also provides specialized calibrant preparations and methods for internal calibration of a MALDI FT-ICR mass spectrometer. The methods (and the devices so prepared) are particularly applicable for use during the rapid acquisition of mass spectra with high mass measurement accuracy on an Fourier transform ion cyclotron resonance mass spectrometer. The methods for internal calibration of a MALDI FT-ICR mass spectrometer include the steps of a) providing a MALDI support that comprises an analyte in a matrix at a first location on the MALDI support, and a calibrant in the matrix at a second location on the MALDI support; b) ionizing the analyte using a laser beam and transiently storing analyte ions in an ion storage chamber; c) ionizing the calibrant using a laser beam and transiently storing calibrant ions in the ion storage chamber; and d) releasing a mixture of analyte ions and calibrant ions from the ion storage chamber into a mass analyzer, thereby providing an internal calibration for the data collected.

Also provided by the present invention are methods and devices for the cost efficient preparation of MALDI supports having a hydrophobic surface and one or more hydrophilic target regions. These hydrophilic/hydrophobic MALDI surfaces are optionally “single use” surfaces on a reusable support; ideally, the sample surfaces can be removed and recreated/regenerated on the same solid support. In addition to the cost effectiveness of the supports, preparation of the sample supports also provides the option of optimizing the surface and the reproducibility of the overall analytical process.

Accordingly, the methods for making a sample support having a hydrophobic surface and one or more hydrophilic target regions include the steps of a) providing a solid support comprising a hydrophobic surface; b) positioning a mask on the hydrophobic surface of the solid support, wherein the mask comprises one or more openings that are positioned at desired locations of the hydrophilic target regions; c) placing the solid support and the mask under reduced air pressure; and d) contacting the desired locations of the hydrophilic target regions with a plasma, wherein the plasma renders the exposed hydrophobic surface under the one or more openings hydrophilic by reactive ion etching, thereby creating the hydrophilic target regions on the hydrophobic surface.

Generation of the hydrophilic target regions can be performed using plasmas generated by various mechanisms, such as radio frequency plasmas, direct current plasmas, or microwave plasmas. Generally, at least 3 W of energy is deposited into the gas during generation of the plasma. In a preferred embodiment, the plasma is an air plasma produced using a radio frequency of at least 500 kHz.

In some embodiments of the methods, treatment of the hydrophobic surface with the plasma produces a hydrophilic functionalized polymer composed of, for example, carboxyl groups, hydroxyl groups, keto groups, epoxide groups, or a combination thereof. For hydrophilic functionalized polymers generated thus and having metal chelating properties, preparation of the MALDI substrate can further include incubating the metal-chelating polymer with one or more metal ions. Alternative embodiments include attachment of various other capture agents via the functionalized surface.

The target substrates of the present invention can also be prepared using previously-used MALDI supports. Regenerating the previously-used support involves a) contacting the support with a plasma and removing organic materials that are attached to the support; and b) contacting one or more surfaces of the support with a hydrophobic derivatizing agent, thereby forming the hydrophobic surface (which can optionally be further treated to generate hydrophilic target regions as described herein). Hydrophobic derivatizing agents for use in the methods of the present invention include any of a number of oils or greases, fluoropolymers, and/or hydrocarbon polymers.

The present invention also provides MALDI sample supports having a hydrophobic surface and one or more hydrophilic target regions, as well as regenerated MALDI sample supports prepared by the methods of the present invention.

Advantageously, the methods and devices of the present invention allow the processing of samples collected, for example, from liquid chromatography. separations, and data acquisition thereof to proceed in fully automated fashion for high throughput LC MALDI FT ICR MS, even for samples containing solvents normally considered unsuitable for MALDI MS.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a surface” includes a combination of two or more surfaces; reference to a “solvent” includes mixtures of solvents, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

As used herein, the term “laser desorption/ionization” (LDI) refers to any of a number of ionization techniques including, but not limited to, matrix assisted LDI (MALDI), IR-MALDI, UV-MALDI, liquid-MALDI, surface-enhanced LDI (SELDI), surface enhanced neat desorption (SEND), desorption/ionization of silicon (DIOS), laser desorption/laser ionization MS, or laser desorption/ two step laser ionization MS.

The terms “solid support,” “substrate,” “solid substrate,” “target substrate,” “MALDI substrate,” “MALDI plate,” “target plate” and the like all refer to the structure or device used to position a sample for interfacing with a laser beam during LDI mass spectrometry.

The term “matrix” as used herein refers to small light absorbing molecules (or surfaces) known in the art, in or on which analytes are dispersed for the purpose of ionization.

The term “MALDI-incompatible solvent” refers to a solvent which interferes with the co-crystallizate formation, ionization, desolvation, and/or other physical processes required for the successful ionization or signal generation process during MALDI MS.

As used herein, the term “depositing” and “spotting” are used interchangeably to refer to the process of placing a sample at a position on, for example, a sample substrate.

The terms “chaotropic agent” or “chaotropic solvent” as used herein refer to solvents or substances capable of specific and/or non-specific interactions with the analytes, thereby affecting or altering the secondary structure of the analytes.

As used herein, the term “sample solubilization agent” refers to a solvent component used to increase or enhance the solubility of one or more sample components.

The term “performance enhancing agent” refers to additives added for cocrystallization in addition to matrix and analyte.

As used herein, the term “analyte” refers to a component of a sample to be analyzed.

The term “calibrant” as used herein refers to one or more compounds of known mass and/or concentration used to the mass scale and/or normalize or calibrate a signal from an instrument, for example, a mass spectrometer signal

The term “mass analyzer” refers to a device used discriminate between ions of different mass-to-charge ratio (m/z) values.

The term “reactive ion etching” refers to a process in which reactive species are produced in a gas (e.g., air, fluorine, chlorine) by electric discharge. The reactive species cause chemical etching of substrates and formation of volatile products. The rate of the etch process is potentially enhanced by substrate surface activation due to ion surface collisions.

### BRIEF DESCRIPTION OF THE DRAWINGS

FIG. 1: Mass spectra of BSA peptide RPCFSALTPDETYVPK (1879.914 Da) from 200 fmol LC-MALDI MS run. Matrix CHCA, DMF multi-step procedure.

FIG. 2: Mass spectra of BSA peptide RPCFSALTPDETVYPK (1879.914 Da) from 200 fmol LC-MALDI MS run. Matrix DHB, teeing procedure.

FIG. 3: Mass spectra of BSA peptide RPCFSALTPDETYVPK (1879.914 Da) from 200 fmol LC-MALDI MS run. Matrix DHB, pre-coat procedure.

FIG. 4: Exemplary embodiment of a mask (panel A), MALDI target plate (panel B), and MALDI target plate holder (panel C) as used in the methods of the present invention.

FIG. 5: MALDI mass spectra after injection, chromatographic separation, and automated deposition of 500 total atomoles of angiotensin II (top) and neurotensin (bottom).

FIG. 6: MALDI mass spectrum of a test mixture of peptides and phosphopeptide before and after enrichment by washing of iron(III) chelated target plates.

### DETAILED DESCRIPTION

Although LC/MS-based approaches for the wide-scale analysis of protein expression patterns exhibit tremendous potential for improved analyses, such approaches typically employ electrospray ionization (ESI) methods. However, while LC/MS experiments using ESI are relatively simple to implement, the operational parameters of ESI coupling methods also impose several limitations. Specifically, the separation system and mass spectrometer employed are coupled directly in real time, making the construction of parallel analysis systems difficult (or at least extremely costly), and often preventing the mass spectrometer from continually collecting useful data due to the equilibration and washing periods typical of separation techniques. More importantly, current instrument control and data analysis software is not nearly fast enough to allow real time data-dependent processing during the course of a chromatographic separation except when employing simple selection criteria such as peak intensity. This necessitates that upon the completion of a separation and subsequent analysis of the resulting data, the same sample must be rerun to focus on those species that exhibited the desired selection criteria. Additionally, monitoring the levels of several particular species over time requires the active engagement of the mass spectrometer over the whole course of the chromatographic run, even though the species of interest themselves elute only in specific narrow time windows throughout the gradient profile. Ultimately, these and other limitations result in dramatic reductions in overall platform throughput.

Alternatively, the information generated by the experiment in real time can be “recorded” by depositing the effluents of the final separation columns directly onto MALDI target plates, thereby creating a permanent record of the multidimensional separation. Decoupling the separation step from the mass spectrometer in this manner guarantees that the chromatography can be performed free of artificially imposed restrictions, while the mass spectrometer can operate at maximum throughput. The resulting plates can also be reanalyzed as desired without the need to repeat the separation step, thus decreasing sample requirements while simultaneously greatly increasing the overall throughput of the system. See, for example, Peters et al. (2002) “An automated LC-MALDI FT-ICR MS Platform for high throughput proteomics” LC•GC Europe July 2002 issue, pp. 2-7.

The sample deposition method employed is critical to the success of creating high fidelity, reproducible “permanent” records of the liquid chromatographic separations. “Heart fractionation” methods (e.g., Griffin et al. (2001) Anal. Chem. 73:978-986) are relatively simple to implement, but potentially sacrifice a significant part of the chromatographic resolution obtained during the separation. By contrast, sophisticated piezo-actuated microdispenser systems fabricated by the anisotropic wet etching of monocrystalline silicon have been described for the deposition of chromatographic effluents directly onto MALDI target plates (see, for example, Ekstrom et al. (2000) Anal. Chem. 72:286-293; Ekstrom et al. (2001) Anal. Chem. 73:214-219). Similarly, specialized liquid junction-coupled sub-atmospheric pressure deposition chambers for the off-line coupling of capillary electrophoresis with MALDI MS have also been described (Preisler et al. (1998) Anal. Chem. 70:5278-5287; Preisler et al. (2000) Anal. Chem. 72:4785-4795). In light of the potential power of this off-line approach, the present invention provides novel methods for preparing samples, and the resulting sample-containing substrate devices. The present invention also provides novel methods for calibrating and analyzing samples. The methods and devices of the present invention are particularly useful, for example, in automated sample processing for direct deposition liquid chromatography MALDI mass spectrometry and high mass measurement accuracy data collection.

MALDI Sample Preparation

The sample preparation process leading to a matrix/analyte co-crystallizate has a profound impact on the signals that are detected from a sample. Only a very limited list of solvents and additives are considered compatible with sample preparation in MALDI MS. However, it is often desirable to use solvents and/or additives that are not MALDI-compatible to, for example, solubilize a sample preparation.

Experimentally, it is found that the sample preparation process leading to a matrix/analyte co-crystallizate directly affects the quality and/or intensity of the signals that are detected from a sample. Standard protocols in use employ a very limited list of solvents and additives that are considered compatible with sample preparation in MALDI MS. Typical solvents include, for example, water and small organic molecules of high volatility (acetonitrile (ACN), acetone, ethanol, methanol, trifluoroacetic acid (TFA), formic acid). A limited number of ammonium salt additives is also used to suppress salt adduct formation. However, it is often desirable to prepare samples using agents that are not compatible with MALDI (e.g., dimethylformamide (DMF), dimethylsulfoxide (DMSO), glycol, polyethylene glycol, glycerol, etc.). For example, strongly chaotropic solvents and/or solvents with low vapor pressures, as well as additives used for sample solubilization (urea, surfactants, salts, etc.) are considered incompatible with MALDI MS. A need exists for sample preparation methods that allow use of such agents but do not prevent the use of MALDI MS.

Accordingly, the present invention provides methods for preparing samples for analysis by MALDI. These methods involve: a) spotting an aliquot of a matrix onto a MALDI support; b) depositing an analyte onto the MALDI support at a same position as the aliquot of the matrix, wherein either the aliquot of matrix or the analyte further comprises a solvents generally considered to be MALDI-incompatible (i.e., a MALDI-incompatible solvent); c) allowing the matrix and analyte to dry, thereby forming a co-crystallizate; d) depositing a recrystallization solution onto the co-crystallizate and redissolving the cocrystallizate; and e) allowing the redissolved co-crystallizate to dry, thereby removing the MALDI-incompatible solvent and forming a sample suitable for MALDI.

MALDI-compatible reagents are known to those of skill in the art. Typical MALDI-compatible solvents include, for example, water and small organic molecules of high volatility, such as acetonitrile (ACN), acetone, ethanol, methanol, trifluoroacetic acid (TFA), formic acid, and the like. MALDI-incompatible reagents, which are also known to those of skill in the art, include, for example, dimethylformamide (DMF), dimethyl sulfoxide (DMSO), N-methylpyrrolidone, methylene chloride, glycol, polyethylene glycol, glycerol, strongly chaotropic solvents, solvents with low vapor pressures, as well as additives used for sample solubilization (urea, surfactants, salts, and the like). However, in the methods of the present invention, either the matrix, the analyte, or both the matrix and the analyte can be prepared in the MALDI-incompatible solvent.

Optionally, the solvents used to prepare the matrix, analyte (or calibrant), or recrystallization solutions can further include a performance enhancing agent. The agents enhance the MALDI process of ionization by, for example, sequestering cations (e.g., such that the protonated form of an analyte molecule is preferably generated over the cationic form). Exemplary performance-enhancing agents include, but are not limited to, ammonium sulfate, diammonium sulfate, diammonium citrate, glucose, and nitrocellulose.

The methods of the invention are useful for a wide variety of matrix components. For example, suitable matrix components include, but are not limited to, α-cyano-4-hydroxycinnamic acid, sinapic acid, 2-(4-hydroxyphenylazo) benzoic acid, succinic acid, 2,6-dihydroxyacetophenone, ferulic acid, caffeic acid, glycerol, 4-nitroaniline, 2,4,6-trihydroxyacetophenone, 3-hydroxypicolinic acid, anthranilic acid, nicotinic acid, salicylamide, trans-3-indoleacrylic acid, dithranol, 2,5-dihydroxybenzoic acid, 3,5-dihydroxybenzoic acid, isovanillin, 3-aminoquinoline, T-2-(3-(4-t-butyl-phenyl)-2-methyl-2-propenylidene)malanonitrile, and 1-isoquinolinol. The matrix can be composed of one or more of these components, and/or a polymer, oligomer, and/or self-assembled monomer of one or more of these matrix components. As understood by one of skill in the art, the matrix chosen for use in the methods of the present invention will depend in part upon the analyte of interest. In some embodiments of the present invention, the matrix employed is a hydrophobic matrix; in other embodiments, a hydrophilic matrix is used. Two preferred matrix/solvent embodiments are α-cyano-4-hydroxycinnamic acid or sinapic acid (4-hydroxy-3,5-dimethoxy-cinnamic acid) prepared in 100% dimethylformamide (a MALDI-incompatible solvent).

In the methods of the present invention, the steps of a) spotting the aliquot of matrix onto the solid support and b) depositing the analyte in the same position can be performed in either order (e.g., by depositing the analyte prior to depositing the matrix). In some embodiments, the sample(s) is/are prepared on a solid support having hydrophilic target regions upon which the matrix and analyte are deposited (see, for example, Schuerenberg et al. (200) Anal. Chem. 72:3436-3442 and products available from Bruker Daltonik (Bremen, Germany); www.bruker-daltonik.de). After deposition of a sample onto a target region, both the analyte and matrix localize into an area equal to or smaller than that occupied by the originally deposited droplet as the sample solvent evaporates, resulting in an increase in effective concentration of the analyte on the hydrophilic anchors. Thus, the use of hydrophilic/hydrophobic sample substrates provides a mechanism for further concentration of the samples after the chromatographic process is complete, enabling the use of 300 μm id capillary columns and commercially available autosamplers. In addition, the localization of analytes to precisely defined locations (e.g., approximately 400 μm in diameter) on the target plates allows the MALDI stage to rapidly move between defined sample locations. Increasing the size of the MALDI laser spot to approximately 400 μm also allows the entire sample to be queried simultaneously, thus greatly reducing the problem of searching for “sweet spots” often encountered when using the dried droplet method of sample preparation. Together, these factors greatly increase the sample throughput of the overall platform.

By dissolving and re-crystallizing the co-crystallizate, the methods of the invention provide a mechanism by which the MALDI-incompatible reagents are removed from the analyte prior to ionization and analysis. Typically, the recrystallization solution includes a MALDI-compatible solvent that can solvate the MALDI-incompatible reagent that is present in the co-crystallizate. In some embodiments, the MALDI-compatible solvent used in the recrystallization solution includes one or more of water, acetonitrile (ACN), acetone, ethanol, methanol, trifluoroacetic acid (TFA), and formic acid. One example of a suitable recrystallization solution is ACN, water, TFA (75/24.9/0.1).

The recrystallization solution can also include, for example, one or more matrix components, which can be either the same or a different matrix than is initially deposited on the MALDI support. Optionally, the recrystallization solution can further include one or more additional components such as a performance-enhancing agent.

The volume of recrystallization solution employed is typically less than or equal to the initial volume of MALDI-incompatible solvent used to transfer the matrix and/or analyte onto the support. Exemplary volumes employed range from 10 μL to sub-microliter volumes (e.g. 100 nL or less). For example, if 10 μL of matrix and solvent is applied to the MALDI substrate, 10 μL, 5 μL or 1 μL of recrystallization solution could be used during the depositing and redissolving steps; if 1 μL of matrix and solvent is applied to the plate, one might use 0.5 μL of recrystallization solution.

In some embodiments of the present invention, the depositing of recrystallization solution and subsequent drying steps are performed multiple times (i.e. repeated) prior to submitting the sample for MALDI mass spectrometry. Particularly in embodiments in which salt is present in the analyte, the methods of the present invention optionally include the step of washing the MALDI support after the co-crystallizate is formed. Washing the MALDI support provides a mechanism for removing (MALDI-incompatible) salts from the co-crystallizates.

The performance of MALDI mass spectrometry can be affected by competitive ionization effects, which are especially prevalent in complex mixtures such as those generated during proteomics studies. The sample preparation methods of the present invention eliminates some of these problems by providing a reproducible environment for the recrystallization of analyte and matrix molecules.

Internal Calibration of MALDI Mass Spectrometer

Yet another area in which MALDI (and other) mass spectrometry has shortcomings for proteomics and other applications is in calibration of the instrument. Any mass spectrometer will perform best in terms of mass measurement accuracy with internal calibration. This is particularly true for Fourier ion cyclotron mass spectrometers, the most accurate mass spectrometers currently available. Despite the inherent accuracy of FT-ICR MS, internal calibration is required to achieve and maintain highest performance under varying acquisition conditions. This means that sample ions (e.g., unknown masses) and calibrant ions (of known masses) need to be measured simultaneously. Usually, this is achieved by ionizing a mixture of sample and calibrant (e.g., a sample that has been spiked with calibrant), and simultaneously submitting the analyte and calibrant ions to the analyzer. However, the spiking process is of questionable utility in MALDI due to the complex processes involved in sample (and calibrant) ionization.

Commonly, analyte/analyte and analyte/calibrant suppression is observed in these spiked mixtures, which can cause the signals of either the analyte or the calibrant to be absent from the spectra, in which case internal calibration fails. Furthermore, since the sample amounts are typically not known before an analysis, the correct amount of calibrant needed to provide for calibration while minimizing signal suppression has to be determined empirically. However, this iterative process is not at all applicable to real time deposited samples in LC-MALDI. Current MALDI targets typically used in automated systems typically have a calibrant location next to every sample location or one calibrant spot in the middle of a set of sample spots to correct for imperfections of the MALDI target, which is an integral part of the analyzer during analysis in time-of-flight (TOF) systems. This improves accuracy but is not to be confused with internal calibration. Preferably, patterns of calibrant and sample locations are avoided because the many calibrant locations reduce the number of samples that fit on a MALDI plate. Also, there is a risk of contamination of samples locations with calibrant during the deposition of calibrant onto calibrant locations either before or after sample deposition.

The present invention provides methods for internal calibration of a mass spectrometer. These methods involve: a) providing a support that comprises an analyte at a first location on the support, and a calibrant at a second location on the support; b) ionizing the analyte and transiently storing analyte ions in an ion storage chamber; c) ionizing the calibrant and transiently storing calibrant ions in the ion storage chamber; and d) releasing the mixture of analyte ions and calibrant ions from the ion storage chamber into a mass analyzer. In a preferred embodiment, the spectrometer comprises a FT-ICR mass spectrometer and the support comprises a MALDI target plate.

These methods reduce the problem of suppression in MS spectra by generating sample ions and calibrant ions from separate sources (e.g., matrix/analyte and matrix/calibrant co-crystallizates used for MALDI). One advantage of this approach is that the maximum sample capacity of the target substrate can be achieved, by positioning the calibrant “outside” the sample area of the plate. Positioning of the calibrants at the outer edges of the target plate also minimizes the risk of contaminating the samples.

Furthermore, the calibration methods of the present invention provide a constant calibrant signal over many experimental runs, thereby providing a controlled and reproducible amount of calibrant ions. This is not typically observed with standard preparations in which the calibrant has been added directly to the sample. In MALDI embodiments of the present invention, standardization of the calibrant signal can be facilitated by optionally applying the matrix/calibrant co-crystallizate as a slurry to selected locations of the sample substrate (e.g., along two edges of the plate), instead of co-crystallizing it at specific locations.

In the calibration methods of the present invention, the steps of ionizing the analyte and ionizing the calibrant (steps b and c) can be performed in either order. In some embodiments of the present invention, the sample support is placed upon an adjustable stage that can be incrementally moved along 2-dimensions (e.g. the x-axis and y-axis with respect to the ionization mechanism). For example, during MALDI, the support is moved such that the analyte and calibrant are sequentially positioned in line with the laser beam. Alternatively, the ionization source can be designed such that the source moves to different positions on the sample support, in line with either the analyte or calibrant deposits.

The methods of the present invention provide for mixing of the analyte and calibrant ions in the gas phase before analysis; typically, this is performed during the transiently storage of the ions in the ion storage chamber. Techniques for trapping and storing ions are known in the art, and include, but are not limited to, the use of one or more multipole ion guides (e.g., quadrupole ion guide, hexapole ion guide, octopole ion guide, stacked ring ion guide), trap electrodes, Penning traps, and the like. Optionally, a combination of ion guides and traps are employed to manipulate the ions during the methods of the present invention. In some embodiments of the methods, the ionized analyte and ionized calibrant are mixed in the analyzer. In preferred embodiments, the analyte and the calibrant ions are allowed to mix and equilibrate in the ion storage chamber prior to being released into the mass analyzer.

In a further embodiment of the methods of the present invention, the ion storage chamber includes an ion trap that provides for mass selection of the trapped ions. The analyte ions, the calibrant ions, or both the analyte and calibrant ions can undergo mass selection during the methods of the present invention. For example, the analyte and calibrant ions are guided using one or more ion optics elements (which could be mass analyzers themselves or possess mass selection capability) into the ion storage chamber, which also optionally has mass selection capabilities. Mass selection is performed during the passage of ions into the ion storage chamber and/or within the ion storage chamber on analyte ions and or calibrant ions.

Method for Making MALDI Target Plates

Another area in which improvements in MALDI MS sample preparation is desirable is the supports used for MALDI. Direct coupling of reverse phase LC (RP-LC) with MALDI MS requires deposition of small portions of the column eluent onto MALDI target plates and the effective mixing of the eluent with a matrix. In some methods in the prior art, the surface of the MALDI target substrate is pre-coated with matrix. However, contamination is a common concern with methods that employ pre-coated target substrates. Moreover, localization must be achieved solely by the way the sample is deposited, and processing options after deposition are very limited.

Accordingly, to facilitate automation of the mass spectral acquisition process, hydrophobic/hydrophilic type MALDI target plates are often employed when the mode of separation is reverse phase-LC (because plates with inverted surface properties are useful in normal-phase LC). Hydrophobic/hydrophilic sample substrates are typically hydrophobic surfaces with a pattern of small hydrophilic regions, or “islands.” Liquid sample fractions deposited onto a hydrophilic island are confined to the location of the hydrophilic island due to surface energy effects. This allows samples to be confined to known locations on the target plate during and after sample processing, with the end result that matrix/analyte co-crystallizates generated by evaporation of solvents will be also located precisely at the island positions. This achieves high sample densities while reducing sample/sample contamination due to mixing of neighboring fractions. Typically, the hydrophobic/hydrophilic (or the reverse patterned hydrophilic/hydrophobic) MALDI target plates are purchased and re-used multiple times. However, the properties of hydrophilic/hydrophobic surfaces degrade through continued use due to exposure to the ionizing plasma, and subsequent cleaning of the surfaces under harsh conditions to remove all sample traces before reuse to prevent cross-contamination. Therefore, a need exists for an efficient means to generate suitable MALDI target plates such that the surfaces can be used once and then regenerated.

The present invention provides methods for making MALDI supports having a hydrophobic surface encompassing one or more hydrophilic target regions. The methods include the steps of: a) providing a solid support having a hydrophobic surface; b) positioning a mask on the hydrophobic surface, wherein the mask comprises one or more openings that are positioned at desired locations of the hydrophilic target regions; c) placing the solid support and the mask under reduced air pressure; and d) contacting the desired locations of the hydrophilic target regions with a plasma, wherein the plasma renders the desired locations (e.g. the hydrophobic regions exposed by the openings in the mask) hydrophilic by reactive ion etching, thereby creating the hydrophilic target regions.

MALDI supports that are suitable for use in the methods of the invention include those that are commercially available. See, e.g., U.S. Pat. No. 6,287,872 to Schurenberg et al, and references cited therein. Masks for use in the methods of the present invention include structures made from aluminum, steel, glass, or any other material that is not substantially effected by the reactive ion etching process. A plurality of openings are typically present on the mask, for generation of the hydrophilic target regions at specified locations. Optionally, the mask has 96, 384, 1536, or 6144 openings. In some embodiments, the mask comprises a sacrificial coating positioned proximal to the hydrophobic surface of the solid support. Typically, the sacrificial coating is provided on an underside of the mask, for placement proximal to the hydrophobic surface. Optionally, the sacrificial coating is composed of the same material as the hydrophobic surface. In one such embodiment, the sacrificial coating further comprises openings aligned with the openings in the mask; placement of the mask and underlying sacrificial coating onto the hydrophobic surface provides an initial “well” into which the plasma can etch (thereby reducing an edge effect of the etching process).

Examples of hydrophobic surfaces include, but are not limited to, a grease film, an oil film, or a synthetic polymer film (such as those available from Cytonix Corporation (Beltsville, Md.) or other self-assembled monolayer of small hydrophobic compound. Most small hydrophobic molecules can be used to generate the hydrophobic surfaces of the present invention. For example, alkanethiols can be used to generate (self-assembled) monolayers on a gold surface. See, for example, Chen et al., (2000) “Using self-assembled monolayers to pattern ECM proteins and cells on substrates” Methods Mol. Biol. 139:209-19; Ulman et al. (2000) “Self-assembled monolayers of rigid thiols” J. Biotechnol. 74(3):175-88; and Whitesides et al. (2001) “Soft lithography in biology and biochemistry” Annu. Rev. Biomed. Eng. 3:335-73.

Hydrocarbon polymers, as well as other hydrophobic classes of polymers not based solely on carbon and hydrogen (e.g., polyphenyleneoxide), or carbon-like analogs such as polysilanes, can be employed as hydrophobic surfaces in the present invention. Exemplary materials which can be used as hydrophobic surfaces in the methods of the present invention include, but are not limited to, PTFE, PTE, PE, PFA, perfluoro alkyaltes and methacrylates, various polysilanes and polysiloxanes optionally substituted with fluoroalkyl groups, and the like. Monolayers can also be prepared from 1H, 1H, 2H, 2H perfluorodecyltrichlorosilane or octadecyltrichlorosilane. In a preferred embodiment, the hydrophobic surface is composed of one or more fluoropolymers, silicones, graphite, graphite filed polymers, polysilanes, and the like. The compositions, alone or in combination, can be applied as a monolayer, a thin-film, a thick-film; they can be applied to a carrier substrate or directly to the substrate itself. Alternately, the solid substrate can be composed or made from these materials.

In addition, various surfaces can be rendered hydrophobic, e.g., by exposure to plasmas containing fluoro compounds. An alternative preferred embodiment of the methods for surface preparation includes the surface fluorination of otherwise unsuitable material by a plasma containing reactive fluoro species like .F, .CF₂, .CF₃, or .SF₅.

The hydrophobic surface can be applied to the solid support in any of a number o methods known to one of skill in the art. For example, distribution of a hydrophobic material (solution of small hydrophobic molecules or a polymer solution) can be achieved by manually tilting the target surface. Polymer solutions can also be applied by spin coating (a common technique used, for example, to disperse photoresist polymer solutions on silicon chips). Alternatively, the hydrophobic surface can be integral to the solid support (e.g., the solid support is manufactured from a material having a hydrophobic nature), in which embodiment, the surface of the solid support directly functions as the hydrophobic surface.

The solid support and mask are placed under reduced air pressure (e.g., a vacuum or partial vacuum) In a preferred embodiment, the air pressure is reduced to 0.3 mbar. However, the methods can be performed at higher or lower air pressures (e.g., 0.1 mbar, 1 mbar, 3 mbar, 10 mbar, etc.).

Generation of the hydrophilic target regions can be performed using various plasmas, such as a radio frequency-generated plasma, a direct current-generated plasma, or a microwave-generated plasma. For example, plasmas for use in the present invention can be produced using air and a radio frequency of at least 500 kHz. Generally, at least 3 W of energy is deposited into the plasma. Furthermore, commercially available units can be employed with specialty gas mixtures and power inputs of hundreds of watts providing higher etch rates. Further details regarding generation and use of plasma for ion reactive etching can be found, for example, in Lieberman and Lichtenberg, Principals of Plasma Discharges and Materials Processing (Wiley, New York, 1994) and Shul and Pearton, Handbook of Advanced Plasma Processing (Springer, New York, 2000).

In some embodiments of the methods, the plasma reacts with the hydrophobic coating to produce a hydrophilic functionalized polymer (e.g., by incomplete oxidation of one or more components of the hydrophobic coating. The hydrophilic functionalized polymer can include carboxyl groups, hydroxyl groups, keto groups, epoxide groups, or combinations of these functionalities. As such, the functionalized surface can be used for various applications.

For example, the newly-derived functionality can be used as a linker or “handle” to further functionalize the surface of the hydrophilic target region with any of a number of moieties, including, but not limited to, various capture agents such as antibodies, ligands, chemically-selective reagents for reactions, and the like. The further functional moieties employed on the surface can be covalently-bound, or the association could be non-covalent (e.g., a salt-bridge formation between carbonyl-functionalized surface and an amine-containing linker or sample component).

Some hydrophilic functionalized polymer produced by these methods were shown to have metal chelating properties. For these embodiments, preparation of the MALDI substrate can optionally further include incubating the metal-chelating polymer with one or more metal ions (e.g., Fe³⁺, Ga³⁺, Zn²⁺, Ni²⁺ or Cu²⁺). Such hydrophilic surfaces can be used for selective binding to sample components, for example during subsequent washing steps. For example, Fe⁺³ and Ga⁺³ when coordinated to certain multivalent ligands (such as iminodiacetic acid or nitrilotriactic acid) exhibit an enhanced affinity for phosphate groups, enabling the selective enrichment of phosphopeptides from a complicated mixture of peptides. Similarly, the coordination of other metals enables the selective enrichment of other functional groups. For example, Ni⁺² can be used for the selective isolation of species containing a 6-His tag. See Raska et al. (2002) “Direct MALDI-MS/MS of phosphopeptides affinity-bound to immobilized metal ion affinity chromatography beads” Anal. Chem. 74(14):3429-33; and Posewitz and Tempst (1999) “Immobilized gallium(III) affinity chromatography of phosphopeptides” Anal. Chem. 71(14):2883-92.

In addition to the solid substrates having a hydrophobic surface and hydrophilic “islands” therein, the reverse arrangement of a hydrophilic surface having hydrophobic islands is also contemplated in the present invention. In one embodiment for preparing a hydrophilic/hydrophobic surface, the mask comprises one or more “patches.” The patches are positioned on the hydrophobic surface above the positions of the desired hydrophobic islands; the plasma then reacts with the remaining exposed regions of the surface, thereby creating a hydrophilic surface having one or more hydrophobic target regions.

In some embodiments, these methods employ MALDI supports that had previously been used for experiments. By regenerating (rather than just reusing) a previously used surface of a plate, one avoids the problems of contamination with sample that is not completely removed from the previous use. These hydrophilic/hydrophobic MALDI surfaces are optionally “single use” sample substrates; ideally, the previously-used sample surfaces can be removed and a fresh surface regenerated on the same solid support. In addition to the cost effectiveness of recycling the supports, control over the preparation of the sample supports also provides the option of optimizing the surface and the reproducibility of the overall analytical process.

Regenerating the previously-used support involves a) contacting the support with a plasma and removing organic materials that are attached to the support; and b) contacting one or more surfaces of the support with a hydrophobic derivatizing agent, thereby forming the hydrophobic surface (which can optionally be further treated to generate hydrophilic target regions as described herein). Optionally, the regenerating process further includes stripping (mechanically and/or through the use of solvents) the surface prior to contacting with the plasma.

The present invention also provides MALDI sample supports having a hydrophobic surface and one or more hydrophilic target regions as prepared by the methods of the present invention. In addition, the present invention provides regenerated MALDI sample supports as described herein, e.g. for use (and reuse) in proteomics studies.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Production of Co-Crystallizates for LC MALDI MS Using a Hydrophobic Matrix

There are recognized difficulties in using commercially-available hydrophobic/hydrophilic plates available from, for example, Bruker Daltonics. In particular, the plates are difficult to use in combination with α-cyano as a matrix (a very commonly used matrix for peptide analysis), especially if performing reversed-phase chromatography (see, for example, Schuerenburg et al. (2000) “Prestructured MALDI-MS sample supports” Anal. Chem. 72(15):3436-42). At lower organic concentrations, the matrix precipitates from solution before localization on the target site is complete. However, optimal co-crystallizates for LC MALDI MS were successfully prepared employing a α-cyano-type hydrophobic MALDI matrix as follows. The MALDI matrix α-cyano-4-hydroxycinnamic acid (CHCA) was dissolved at a concentration of 1.0 mg/mL in 100% DMF. Of this solution, 75 nL was deposited by a deposition system onto each of the hydrophilic islands (circular 400 μm diameter areas) of a hydrophobic/hydrophilic MALDI target.

For analysis, 200 fmol of a BSA digest was separated by liquid chromatography. The eluent of a micro-LC column running at a flow rate of 3 μL/min was fraction collected (0.5 μL fractions) every 10 seconds onto the hydrophilic islands spotted, or “charged,” with the matrix solution. The mixtures of matrix solution and eluent were allowed to shrink and fully dry onto the hydrophilic islands. Premature precipitation of matrix out of the highly aqueous early fractions is prevented by the presence of the strong relatively nonvolatile DMF.

After drying the co-crystallized matrix and eluent, the co-crystallizates were recrystallized on the target substrate by charging each island with 35 nL (e.g., a volume just covering the island) of a solution of 0.2 mg/ml of CHCA in ACN, Water, TFA (75/24.9/0.1). The samples were ready to be analyzed by MALDI MS after having dried down again.

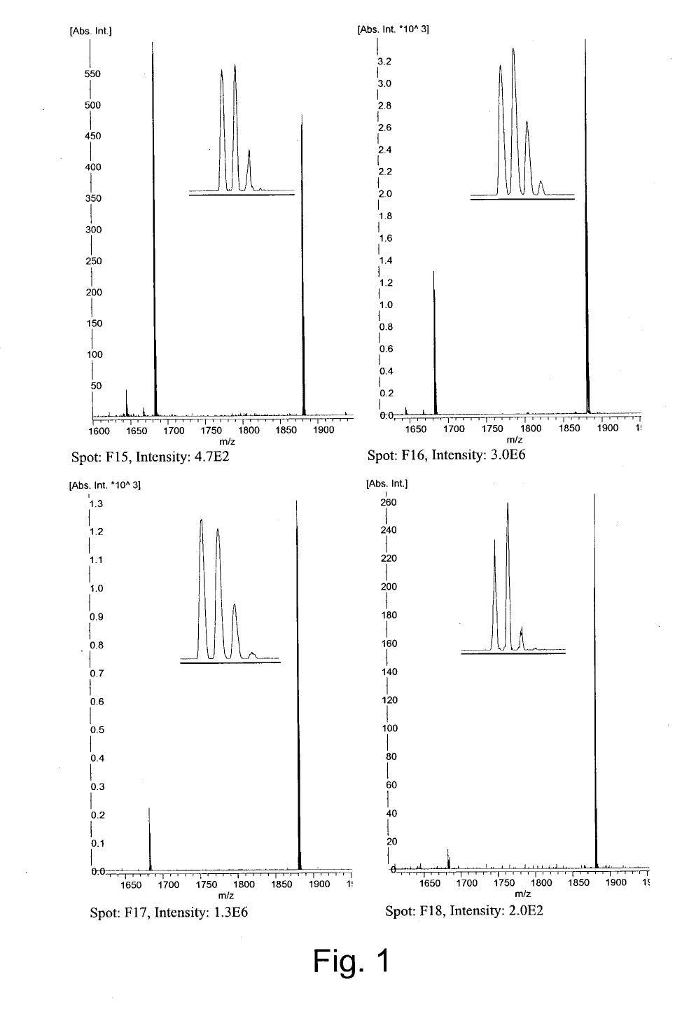

FIG. 1 shows selected data for the peptide RPCFSALTPDETYVPK (mass 1879.914 Da) from bovine serum albumin (BSA). This example demonstrates the great sensitivity of this method for a hydrophobic MALDI matrix.

### Example 2

### Production of Co-Crystallizates for LC MALDI MS Using a Hydrophilic Matrix

An example of a method for the production of optimal co-crystallizates for LC MALDI MS employing a hydrophilic matrix consists of the following steps. For analysis, the BSA digest was fractionated by liquid chromatography. Prior to deposition on the target support, A 3 mg/ml solution of 3,5 dihydroxybenzoic acid (DHB) in water was merged (“teed”) at a flow rate of 4 μL/min into the eluent of the micro-LC column running at a flow rate of 3 μl/min. The fractions were deposited by a deposition system onto the hydrophilic islands (circular 400 μm diameter areas) of a hydrophobic/hydrophilic MALDI target plate every 10 seconds (1.17 μL a fraction) and left for air drying without further processing before analysis.

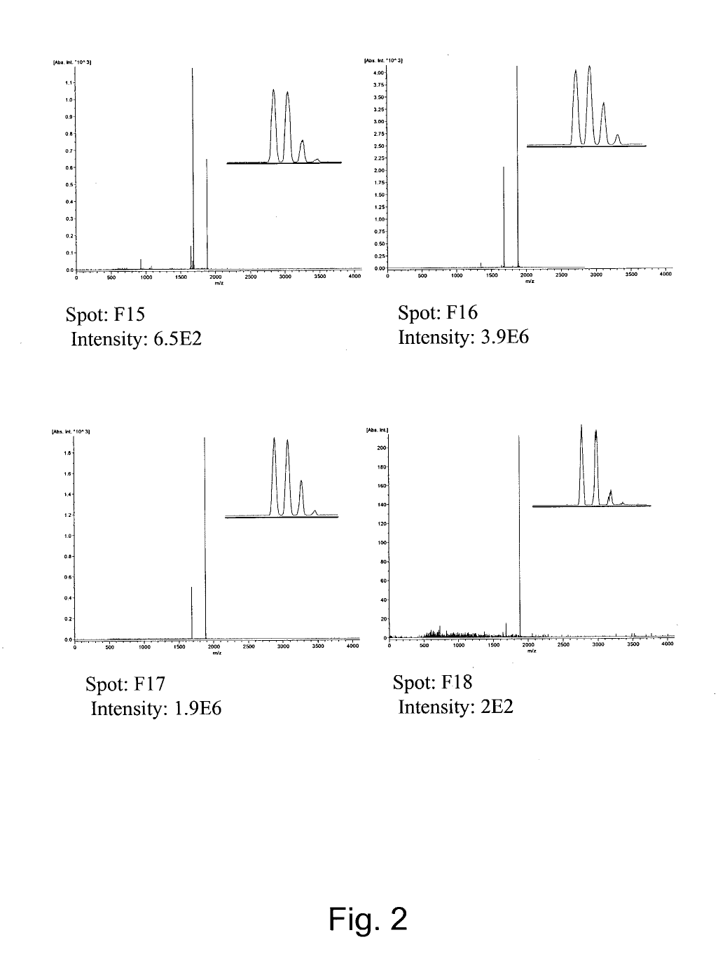

FIG. 2 shows selected data for the peptide RPCFSALTPDETYVPK of mass 1879.914 Da from bovine serum albumin (BSA). For analysis, 200 fmol of a BSA digest was separated by liquid chromatography, and fraction collected onto a hydrophobic/hydrophilic target every 10 seconds. This example demonstrates the great sensitivity of this method for a hydrophilic MALDI matrix.

### Example 3

### Production of Co-Crystallizates for LC MALDI MS Using an Alternate Hydrophilic Matrix

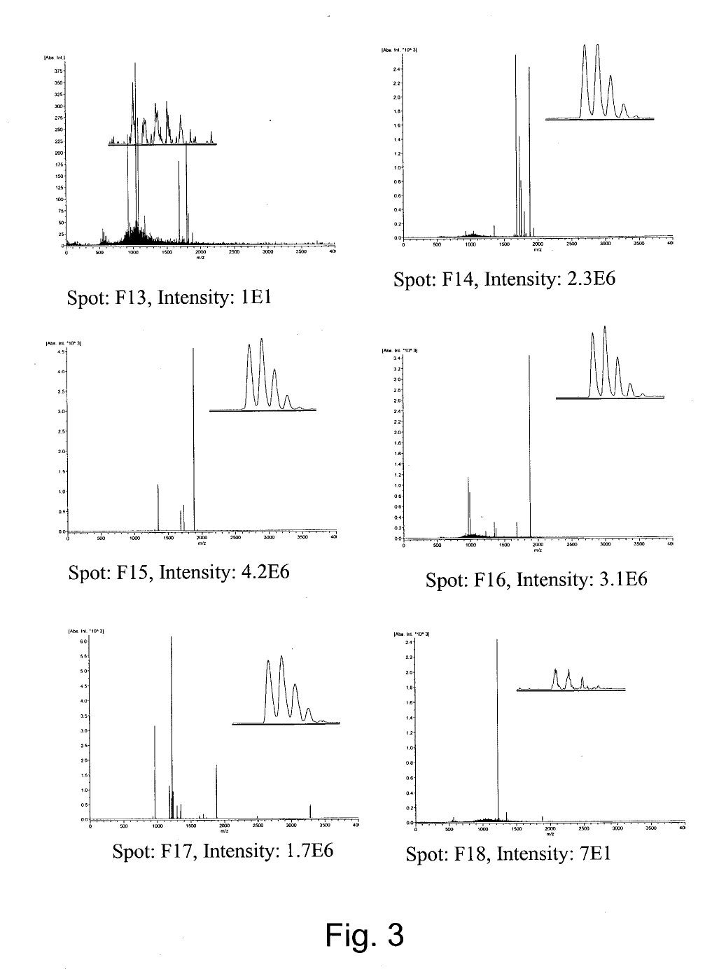

A second example of a method for the production of co-crystallizates for LC MALDI MS employing a hydrophilic matrix consists of the following steps. Hydrophilic islands of a MALDI target were charged with 0.66 μl of a 3 mg/ml solution of DHB in water by a deposition system at a rate of 1 Hz. The eluent of a micro-LC column running at a flow rate of 3 μL/min was fraction collected (0.5 μL fractions) every 10 seconds onto the hydrophilic islands spotted, or “charged,” with the matrix solution, and left to air dry. The formed co-crystallizates are recrystallized on the target by charging each island with 0.3 μl of a ACN, water, TFA (50/49.9/0.1) solution. Sample fractions are left to air dry before analysis. FIG. 3 shows selected data for the peptide RPCFSALTPDETYVPK of mass 1879.914 Da from bovine serum albumin (BSA). For analysis, 200 fmol of a BSA digest was separated by liquid chromatography, fraction collected onto a hydrophobic/hydrophilic target every 10 seconds, and recrystallized according to the above method. This example further demonstrates the great sensitivity obtained with this method for a hydrophilic MALDI matrix.

### Example 4

### Preparation of Calibrant-Containing Sample Plates

MALDI target plates having matrix/calibrant applied at specified positions are prepared as follows. A saturated solution of matrix in a solution of ACN+water+TFA (80/19.9/0.1) was prepared by vortexing excess matrix in 1 mL of the solution for 5 minutes. Undissolved matrix was removed by centrifugation, and the supernatant was collected into a microcentrifuge tube. A 10⁻⁵ M solution of calibrants in the ACN+water+TFA (80/19.9/0.1) solution was prepared. 100 μL of the calibrant solution was added to the supernatant of the matrix solution and vortexed to mix. The microcentrifuge tube containing the matrix and calibrants was then placed in a rotary evaporator and brought to complete dryness. The matrix/calibrant co-crystallizate formed in such a fashion was then crushed by adding approximately 5 to 10 stainless steel balls (1.5 mm diameter) to the tube and vortexing for 20 minutes. A slurry of crushed matrix/calibrant co-crystallizate was prepared in 0.5 mL of hexanes and applied with a pipette to desired locations on the MALDI target. The hexanes solvent was evaporated, leaving a homogeneous fine crystalline matrix/calibrant co-crystallizate behind.

### Example 5

### Internal Calibration Methods

The calibration methods of the present invention have been performed during MALDI experiments using the sample- and calibrant-containing target substrates as prepared by the methods of the present invention. The prepared target plate was mounted onto linearly encoded high precision x- and y-stages in a custom-built intermediate pressure MALDI source. Sample or calibrant ions were generated by positioning either the sample locations (e.g., first positions) or calibrant strips (e.g., second positions) within the focal point of a UV-laser beam. The analyte and calibrant ions were collisionally cooled by the surrounding nitrogen buffer gas and guided by a first quadrupole (cooling quadrupole) to the entrance of a second quadrupole (selection quadrupole), through which they were passed into a hexapole ion guide for transient storage. The selection quadrupole can be operated in integral or mass selective mode, allowing for the optional isolation of an individual species prior to ion accumulation in the hexapole.

To ensures the high mass accuracy of the data collected by FT-ICR MS, internal calibration was performed using a novel gas phase mixing scheme. After a sample at a first position was irradiated with the UV laser and the resulting ions were stored (e.g., in the hexapole), the sample stage rapidly moves (on the order of a fraction of a second) to a second position containing the calibrant. Typically, the calibrant is placed in a “strip” along both edges of the target plate; as such, the second position of calibrant selected by the sample stage is the closer of the two calibrant strips on the side of the plates. After irradiation of the calibrant strip, the calibrant ions were mixed with the ions from the sample in the hexapole. The mixture of sample (analyte) ions and calibrant ions were transferred through the remaining ion optics into the mass analyzer, which is located inside the bore of a 7 T superconducting magnet. Thus, internal calibration is achieved without added calibrant directly to the samples, thereby preserving the sample integrity.

After transient storage, the ions were transferred to the analyzer cell, cooled by gas injection, and detected. The data acquisition process is controlled by customized proprietary software. The total time required for the acquisition of a typical mass spectrum is roughly 7 to 10 seconds, mostly due to the time required for pump down after gas injection (2 s) and the acquisition of an one million points time domain signal (˜3.5 s). Thus, several hundred internally calibrated mass spectra of samples fractions can be acquired in less than 1 hr, without contaminating the sample fractions with calibrant.

### Example 6

### Preparation and Use of Regenerated MALDI Target Plates

The preparation of a fresh hydrophilic/hydrophobic surface on a MALDI target plate consists of the following steps. For this example, a used electro-polished microtiter-sized stainless steel MALDI target plate was employed; however, the process can also be used for preparation of new target plates.

The surface coating of the used MALDI sample substrate was mechanically stripped from the surface of the plate, along with any residual sample and calibrant co-crystallizates, by wiping the surface with acetone. The mechanically-stripped plate was further cleaned by sonication in 50% isopropanol for approximately 30 minutes, followed by a 30 minute exposure to a radio-frequency air plasma (described below) to remove any remaining organic materials by oxidation to gaseous compounds.

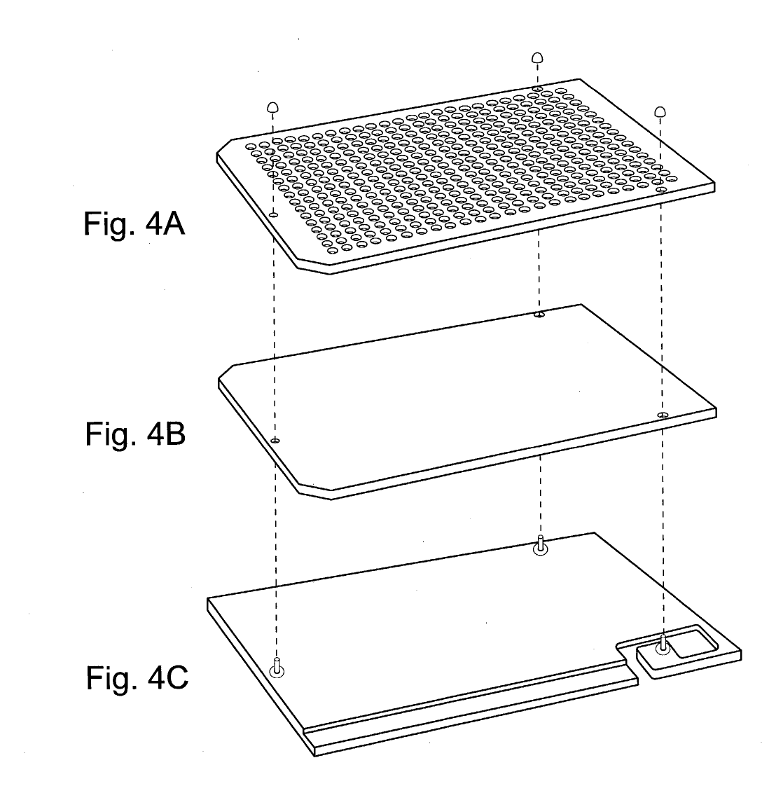

After plasma cleaning, a 1 mL aliquot of a fluoropolymer solution (FluroroPel PFC 1601V/FS, Cytonix Corp., Maryland) was pipetted onto the cleaned surface. Uniform distribution was achieved by manually tilting the plate; the plate was then vertically mounted for air drying, during which the excess solution was allowed to run off the edge of the target substrate. After air drying, the substrate was baked for approximately 30 minutes in a convection oven at 160° C. After cooling, the plate was mounted onto a metal holder to facilitates the accurate positioning of an aluminum mask (see FIG. 4. The mask employed is chosen based upon the intended use of the plate; masks having, for example, 384, 1536, or 6144 openings, each opening having at least a 350 μm diameter. The selected mask was placed on the fluoropolymer coated (top) surface. The stack of holder, plate, and mask was placed inside a vacuum box. The vacuum box was evacuated via a mechanical pump, then the pressure inside the box was adjusted to 0.3 mbar by bleeding air into the container.

An estimated 8 W of energy at a frequency of 2 MHz was deposited into the plasma by the driver. Hydrophilic islands of a diameter corresponding to that of the mask openings (e.g., 350 μm) were formed by reactive ion etching (RIE) during long exposures (30 minutes) to the air plasma by fully removing the coating and exposing the steel surface. Shorter exposures (30 seconds) leave an oxidized hydrophilic polymer surface. Plates manufactured in this fashion were used to produce the data in FIGS. 1-3.

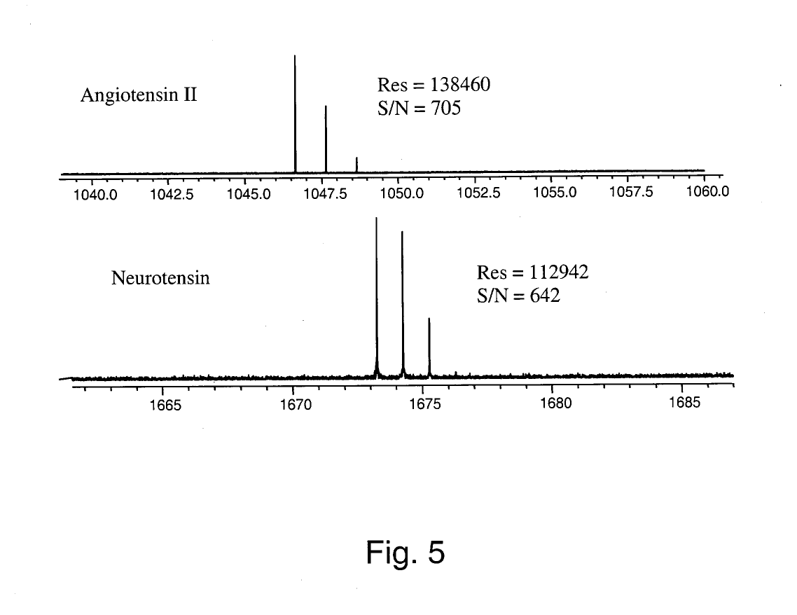

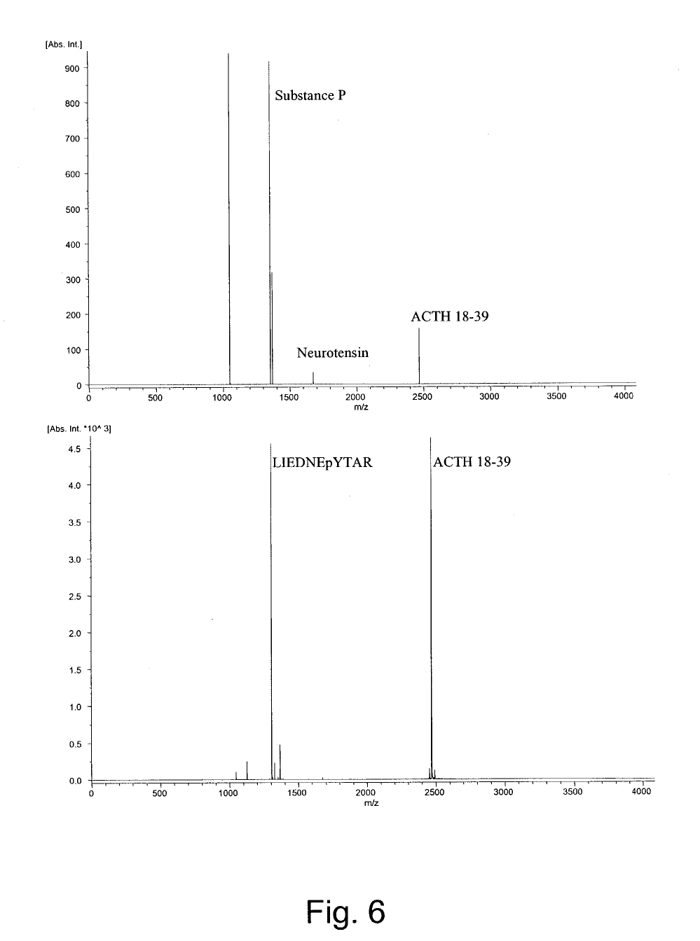

Hydrophilic polymer surfaces were produced during shorter exposures to plasma; these surfaces were shown to possess metal chelating properties and are useful in the enrichment of samples with high affinities to metal chelates. An example of an enrichment experiment of this type consists of the following steps. A MALDI plate was produced according to the procedure given above for which the plasma etch time was shortened to 30 seconds. The plate was incubated with 100 mM FeCl₃ solution for 1.5 hrs, washed with water and left to dry. A test mixture of the peptides angiotensin II, substance P, neurotensin, ACTH 18-39, and a phosphopeptide (LIEDNEpYTAR) was applied to the hydrophilic target regions of the plate and left to dry. A portion of the hydrophilic target regions were washed twice with a 100 mM solution of NaCl, 1% acetic acid, 25% ACN, followed by two washes with 0.1% acetic acid. Spectra for the washed and unwashed samples were recorded after application of matrix. As seen in FIG. 5, the phosphopeptide signal is not observed in the unwashed sample (as expected due to suppression). Selective enrichment of the signals derived from the washed hydrophilic regions of the MALDI plate allows the phosphopeptide to become clearly visible in the washed sample (FIG. 6).

### Example 7

### Automatable LC-MALDI Mass Spectrometry Platforms

The process for preparing the matrix/analyte co-crystallizate for MALDI MS has a profound impact on the signals that are detected from the sample. Preparation and handling of the sample

prior to deposition on the sample substrate also can affect signal generation. Gradient elution from an LC column causes the eluent composition to change (for example, from aqueous to organic) in the course of a run. Samples collected from gradient LC as such are not suitable for MALDI MS, due to several inherent problems, including: a) co-crystallizate formation does not occur under uniform conditions for all fractions due to changes in the solvent composition, b) sample/matrix deposition is impaired due to the limited solubility of the commonly-used hydrophobic matrices in aqueous solutions at the start of the run, and c) the highly organic solvent mixture eluting at later stages of the run cause incomplete sample localization due to the enhanced wetting properties on the surface of the sample substrate. While the sample substrates of the present invention address these issues in part, methods of sample preparation that eliminates the effects of gradient elution on the properties of the cocrystallizate can also employed.

The sample plates prepared by the methods of the preset invention are particularly useful in automated sample processes for direct deposition liquid chromatography MALDI mass spectrometry and high mass measurement accuracy data collection. Optionally, samples having a plurality of analytes are initially fractionated using either chromatographic methods (such as strong cation exchange or immobilized metal affinity) or amino acid specific enrichment techniques . The sample (either intact or as fractionated components) is loaded onto one or more reverse-phase μHPLC columns. MALDI has been shown to preferentially promote the ionization of more hydrophobic peptides in complicated mixtures due to their enhanced co-crystallization with the matrix, thus providing an incomplete representation of a sample's composition. However, by subjecting the peptide mixtures to reversed-phase μHPLC immediately before deposition, all of the peptides deposited in an individual spot should exhibit nearly identical hydrophobicities, and therefore co-crystallize more similarly without resorting to complicated recrystallization methods.

The outlets of the μHPLC columns are positioned in parallel, and MALDI target plates clamped to an automated x,y translational stage are physically moved beneath the columns, allowing for the concomitant deposition of a chromatographic run and matrix onto a MALDI target plate. Preferably, the outlets of the columns and the target plates do not come into direct physical contact (see, e.g., International Patent Application No. PCT/US02/01536, filed Jan. 17, 2002). Rather, the eluents of the columns are transferred to the plates using, for example, a charge induction mechanism by applying a constant or intermittent negative potential to the target plates. This sample transfer mechanism results in either a continuous stream or a series of droplets of precisely controlled volume respectively. The deposition of a continuous stream potentially would more accurately maintain the fidelity of the separation process. However, such an approach would also require the MALDI system to query the entire length of the trace, since eluting samples would not be localized to any particular position.

During sample deposition, roughly 150 nL volume aqueous droplets can be precisely arrayed on a three by five square inch stainless steel target plate in a 6144 microtiter array format, with each spot clearly distinguished from its nearest neighbors. Using this deposition system, matrix can also automatically be applied to the target plate before, during, or after the chromatographic process as described herein. Additionally, the deposition system works equally well with aqueous or numerous organic solvents, enabling the recrystallization processes described herein and thus no longer limiting sample deposition procedures to MALDI-compatible solvent mixtures (such as acetonitrile and water). The unique combination of automation and operational flexibility enables the optimization of the many variables known to effect MALDI crystallization processes.

FIG. 5 shows the quality of the signals obtained after the injection of 500 total atomoles of each analyte peptide, automated reversed-phase μHPLC and sample plate deposition, and automated mass spectrometric analysis using a custom-built MALDI source and a highly modified commercial FT-ICR MS. Although the “permanent” nature of the deposited MALDI sample enables the extended accumulation of signal for low concentration species, these signals were obtained from only 100 laser shots in a single accumulation event. Such excellent signals are routinely obtained in less than 7 seconds after a fully automated sample preparation and analysis process, demonstrating the system's capability for rapid, high-sensitivity sample throughput.

Uses of the Methods and Devices of the Present Invention

The sheer complexity and temporal nature of the proteome necessitates the development of more powerful, higher-throughput analysis platforms. MALDI-based LC/MS platforms employing the methods and devices of the present invention (as well as the methods, compositions, and devices of copending U.S. application Ser. No. ______ [GNF Docket No. P0051US30 and Ser. No. ______ Attorney Docket No. 36-002030US]) address many of the operational disadvantages inherent in the real time coupling of separation systems and mass spectrometers using ESI ionization. The sample processing methods described herein are designed to maximize the throughput of any MALDI-based analytical platform, and are therefore compatible with a wide range of commercially available instrumentation.

Modifications can be made to the method and materials as described above without departing from the spirit or scope of the invention as claimed, and the invention can be put to a number of different uses, including the use of any method herein, to prepare a sample and/or calibrant for MALDI MS, and the use of any method herein, to prepare a sample substrate for use in MALDI MS. In a further aspect, the present invention provides for the use of any sample substrate or component herein, for the practice of any method or assay herein.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for preparing a sample for matrix-assisted laser desorption/ionization (MALDI) spectrometry, the method comprising:
spotting onto a MALDI support an aliquot of a matrix;
depositing an analyte onto the MALDI support at a same position as the aliquot of the matrix, wherein either the aliquot of matrix or the analyte further comprises a MALDI-incompatible solvent;
allowing the matrix and analyte to dry onto the MALDI support to form a cocrystallizate;
depositing onto the co-crystallizate a recrystallization solution into which the cocrystallizate redissolves; and
allowing the redissolved co-crystallizate to dry, thereby forming a sample suitable for MALDI.

2. The method of claim 1, wherein the matrix comprises a component selected from the group consisting of α-cyano-4-hydroxycinnamic acid, sinapic acid, 2-(4-hydroxyphenylazo) benzoic acid, succinic acid, 2,6-dihydroxyacetophenone, ferulic acid, caffeic acid, glycerol, 4-nitroaniline, 2,4,6-trihydroxyacetophenone, 3-hydroxypicolinic acid, anthranilic acid, nicotinic acid, salicylamide, trans-3-indoleacrylic acid, dithranol, 2,5-dihydroxybenzoic acid, 3,5-dihydroxybenzoic acid, isovanillin, 3-aminoquinoline, T-2-(3-(4-t-butyl-phenyl)2-methyl-2-propenylidene)malanonitrile, and 1-isoquinolinol.

3. The method of claim 2, wherein the matrix comprises a polymer, oligomer, or self-assembled monolayer of one or more of the components.

4. The method of claim 1, wherein the MALDI-incompatible solvent comprises a chaotropic agent.

5. The method of claim 1, wherein the MALDI-incompatible solvent comprises a solvent having a low vapor pressure.

6. The method of claim 1, wherein the MALDI-incompatible solvent comprises dimethylformamide, dimethylsulfoxide, N-methylpyrrolidone, methylene chloride, polyethylene glycol, glycol or glycerol.

7. The method of claim 1, wherein the matrix is a hydrophobic matrix and the MALDI-incompatible solvent is one that can solvate the matrix.

8. The method of claim 7, wherein the matrix is α-cyano-4-hydroxycinnamic acid or sinapic acid and the MALDI-incompatible solvent comprises 100% dimethylformamide.

9. The method of claim 1, wherein the matrix is a hydrophilic matrix and the MALDI-incompatible solvent is one that can solvate the analyte.

10. The method of claim 1, wherein the MALDI-incompatible solvent comprises a sample solubilization agent.

11. The method of claim 10, wherein the sample solubilization agent comprises one or more compounds selected from the group consisting of urea, a surfactant, and a salt.

12. The method of claim 1, wherein the MALDI support comprises hydrophilic target regions upon which the aliquots of the matrix are spotted.

13. The method of claim 1, wherein spotting the aliquot of the matrix onto the MALDI support is performed prior to depositing the analyte onto the MALDI support.

14. The method of claim 1, wherein depositing the analyte onto the MALDI support is performed prior to spotting the aliquot of the matrix onto the MALDI support.

15. The method of claim 1, wherein the analyte and the aliquot of the matrix are co-deposited onto the MALDI support.

16. The method of claim 1, wherein the recrystallization solution comprises a MALDI-compatible solvent.

17. The method of claim 16, wherein the MALDI-compatible solvent comprises one or more of water, acetonitrile (ACN), acetone, ethanol, methanol, trifluoroacetic acid (TFA), and formic acid.

18. The method of claim 17, wherein the MALDI-compatible solvent comprises a solution of 75% ACN, 24.9% water and 0.1% TFA by volume.

19. The method of claim 16, wherein the recrystallization solution further comprises one or more matrix components, a performance enhancing agent, or a combination thereof.

20. The method of claim 1, wherein the method further comprises:
washing the MALDI support prior to depositing the recrystallization solution onto co-crystallizate.

21. The method of claim 1, wherein a volume of the recrystallization solution deposited onto the co-crystallizate is less than a volume of the aliquot of matrix and MALDI-incompatible solvent.

22. The method of claim 21, wherein the volume of the recrystallization solution deposited onto the co-crystallizate is less than 10 μL.

23. The method of claim 21, wherein the volume of the recrystallization solution deposited onto the co-crystallizate is less than 5 μL.

24. The method of claim 21, wherein the volume of the recrystallization solution deposited onto the co-crystallizate is less than 1 μL.

25. The method of claim 21, wherein the volume of the recrystallization solution deposited onto the co-crystallizate is less than 0.5 μL.

26. The method of claim 21, wherein the volume of the recrystallization solution deposited onto the co-crystallizate is less than 100 nL.

27. The method of claim 1, wherein the method further comprises repeating one or more times the steps of depositing the recrystallization solution and allowing the redissolved cocrystallizate to dry.

28. A sample on a sample support for MALDI mass spectrometry as prepared by the method of claim 1.

29. A method for internal calibration of mass data generated by a mass spectrometer, the method comprising:
a) providing a support that comprises an analyte at a first location on the support, and a calibrant at a second location on the support;
b) ionizing the analyte and transiently storing analyte ions in an ion storage chamber;
c) ionizing the calibrant and transiently storing calibrant ions in the ion storage chamber; and
d) releasing a mixture of analyte ions and calibrant ions from the ion storage chamber into a mass analyzer.

30. The method of claim 29, wherein step b) is performed prior to step c).

31. The method of claim 29, wherein step c) is performed prior to step b).

32. The method of claim 29, wherein the second location on the support comprises one or more side regions of the support.

33. The method of claim 29, wherein transiently storing the analyte ions and the calibrant ions comprises trapping the ions with one or more multipole ion guides, trap electrodes, Penning traps, or a combination thereof.

34. The method of claim 33, wherein the one or more multipole ion guides comprise a quadrupole ion guide, a hexapole ion guide, a octopole ion guide, a stacked ring ion guide, or a combination thereof.

35. The method of claim 29, wherein the ion storage chamber comprises an ion trap that provides for mass selection.

36. The method of claim 35, wherein mass selection is performed during ion transport into the ion storage chamber.

37. The method of claim 29, wherein transiently storing the analyte ions and the calibrant ions comprises guiding the ions using one or more ion optics elements to an entrance of a first mass analyzer, and passing the ions through the first mass analyzer into the ion storage chamber.

38. The method of claim 37, wherein passing the analyte ions or the calibrant ions through the first mass analyzer further comprises performing mass selection on the ions.

39. The method of claim 29, wherein providing the support comprises placing the support on a movable x-y-stage; and wherein ionizing the analyte and ionizing the calibrant comprises moving the support to sequentially position the analyte and the calibrant in line with a laser beam.

40. The method of claim 29, wherein ionizing the analyte and ionizing the calibrant comprises sequentially moving a laser beam in line with the analyte at the first position and the calibrant at the second position.

41. A method for internal calibration of a mass spectrometer, the method comprising:
a) ionizing an analyte by a first method of ionization, transporting the resulting analyte ions into an ion storage chamber through a first set of ion optical elements, and transiently storing the analyte ions in the ion storage chamber;
b) ionizing a calibrant by a second method of ionization, transporting the resulting calibrant ions into an ion storage chamber through a second set of ion optical elements, and transiently storing the calibrant ions in the ion storage chamber; and
c) releasing the mixture of calibrant and analyte ions into a mass analyzer.

42. The method of claim 41, wherein step a) is performed prior to step b).

43. The method of claim 41, wherein step b) is performed prior to step a).

44. The method of claim 41, wherein ionizing the analyte and ionizing the calibrant comprise performing ESI.

45. The method of claim 41, wherein ionizing the analyte and ionizing the calibrant comprise performing MALDI.

46. The method of claim 41, wherein ionizing the analyte and ionizing the calibrant comprise performing different ionization techniques.

47. The method of claim 41, wherein the first and second sets of ion optical elements comprise a same set of ion optical elements.

48. The method of claim 41, wherein the ion storage chamber comprises an ionization region of an ion source.

49. The method of claim 41, wherein the mass spectrometer comprises a FT-ICR mass spectrometer.

50. The method of claim 41, wherein ionizing the analyte and ionizing the calibrant comprises positioning an ion source proximal to the first or second set of ion optical elements.

51. A method for making a sample support having a hydrophobic surface having one or more hydrophilic target regions, the method comprising:
providing a solid support comprising a hydrophobic surface;
positioning a mask on the hydrophobic surface, wherein the mask comprises one or more openings that are positioned at desired locations of the hydrophilic target regions;
placing the solid support and the mask under reduced air pressure; and
contacting the desired locations of the hydrophilic target regions with a plasma, wherein the plasma renders the hydrophobic surface under the one or more openings in the mask hydrophilic by reactive ion etching, thereby creating the hydrophilic target regions on the hydrophobic surface.

52. The method of claim 51, wherein providing the solid support comprises exposing the surface to a plasma comprising one or more fluoro compounds.

53. The method of claim 51, wherein the hydrophobic surface comprises PTFE, PTE, PE, PFA, graphite, a monolayer prepared from 1H, 1H, 2H, 2H perfluorodecyltrichlorosilane, a monolayer of octadecyltrichlorosilane, perfluoro alkylate, perfluoromethacrylate, polysilane, polysiloxane, fluoroalkyl-substituted polysilane., or fluoroalkyl-substituted siloxane.

54. The method of claim 51, wherein the plasma comprises a radio frequency-generated plasma, a direct current-generated plasma, or a microwave-generated plasma.

55. The method of claim 54, wherein the plasma is an air plasma.

56. The method of claim 51, wherein the mask comprises 96, 384, 1536, or 6144 openings.

57. The method of claim 51, wherein the mask comprises a sacrificial coating positioned proximal to the hydrophobic surface of the solid support.

58. The method of claim 51, wherein contacting the desired locations of the hydrophilic target regions with the plasma further comprises depositing at least 3 W of energy into the plasma.

59. The method of claim 51, wherein contacting with the plasma further comprises generating the plasma using a radio frequency of at least 500 kHz.

60. The method of claim 51, contacting the desired locations of the hydrophilic target regions with the plasma further comprises generating a hydrophilic functionalized polymer comprising carboxyl groups, hydroxyl groups, keto groups, epoxide groups, or a combination thereof.

61. The method of claim 60, wherein the hydrophilic target regions comprise a metal chelating polymer.

62. The method of claim 61, further comprising incubating the hydrophilic target regions with one or more metal ions.

63. The method of claim 62, wherein the one or more metal ions are selected from the group consisting of Fe³⁺, Ga³⁺, Zn²⁺, Ni²⁺ and Cu²⁺.

64. The method of claim 60, further comprising incubating the hydrophilic target regions with one or more capture agents capable of associating with the hydrophilic target regions.

65. The method of claim 64, wherein the one or more capture agent comprises an antibody.

66. The method of claim 51, wherein providing the solid support comprises regenerating a previously-used hydrophilic/hydrophobic MALDI support.

67. The method of claim 66, wherein regenerating the previously-used support comprises:
contacting one or more surfaces of the support with a plasma and removing organic materials that are attached to the support; surface and
contacting the support surface with a hydrophobic derivatizing agent, thereby forming a hydrophobically-derivatized surface and regenerating the previously-used support.

68. The method of claim 67, further comprising mechanically or chemically stripping the support prior to contacting with the plasma.

69. The method of claim 67, wherein the plasma comprises a radio frequency-generated plasma, a direct current-generated plasma, or a microwave-generated plasma.

70. The method of claim 67, wherein the hydrophobic derivatizing agent comprises an oil or grease film.

71. The method of claim 67, wherein the hydrophobic derivatizing agent comprises one or more of a fluoropolymer and a hydrocarbon polymer.

72. The method of claim 71, wherein the hydrophobic derivatizing agent comprises a 1-4% fluoropolymer solutions in a fluorosolvent.

73. The MALDI sample support having a hydrophobic surface and one or more hydrophilic target regions as prepared by the method of claim 51.

74. The regenerated MALDI sample support as prepared by the method of claim 67.
